(19) United States
(12) Patent Application Publication
(10) Pub. No.: US 2024/0212794 A1
(43) Pub. Date: Jun. 27, 2024

(54) METHOD FOR CONSTRUCTING A MODEL SIMULATING A CHEMICAL REACTION

(71) Applicant: IFP Energies nouvelles, RUEIL-MALMAISON (FR)

(72) Inventors: Cedric MEHL, RUEIL-MALMAISON CEDEX (FR); Thibault FANEY, RUEIL-MALMAISON CEDEX (FR)

(21) Appl. No.: 18/555,771

(22) PCT Filed: Apr. 22, 2022
(86) PCT No.: PCT/EP2022/060739

§ 371 Date: Oct. 17, 2023

(30) Foreign Application Priority Data
May 5, 2021 (FR) FR2104735

(51) Int. Cl. G16C 20/10; G16C 20/70

(52) U.S. Cl. CPC G16C 20/10; G16C 20/70

(57) ABSTRACT

The present invention is a method of building a simulation model (MSI) of at least one chemical reaction, which builds an intermediate model using an artificial neural network (RNA) and a training base (BAP), and then builds the simulation model by adding an additional mass conservation layer (RNC) to artificial neural network (RNA). Furthermore, the invention relates to a method of simulating (SIM) a chemical reaction implementing the simulation model (MSI).

### CROSS-REFERENCE TO RELATED APPLICATIONS

Reference is made to PCT/EP2022/060739 filed Apr. 22, 2022, and French Patent Application No. 2104735 filed May 5, 2021, which are incorporated herein by reference in their entirety.

### BACKGROUND OF THE INVENTION

### Field of the Invention

The present invention relates to the field of chemical kinetics simulation, for predicating a chemical species mixture at the outlet of a reactive system.

### Description of the Prior Art

Predicting behavior of chemical reactive systems is important for designing many industrial systems, such as energy conversion methods or systems which provide chemical propulsion transportation, and for problems related to the transportation of these chemical compounds. In these systems, a fluid containing a mixture of chemical species undergoes various transformations referred to as chemical reactions. For example, it may be the prediction of the composition of chemical species occurring in combustion, notably in a combustion engine, of chemical reactions in an electrical battery, in a fuel cell, in a reactive flow, in a catalytic process, etc.

This modeling predicts the evolution of chemical reagents (chemical species) in a reactive system between an inlet and an outlet. The notion of inlet and outlet can correspond to one of the following: the inlet and the outlet can correspond to a physical inlet and outlet of a system (before and after a chemical reaction), and to the evolution of the system between two time intervals during a numerical simulation (before or after a time interval of a chemical reaction).

So-called data-based approaches can be used to simulate chemical reactions. Such approaches allow prediction of the behavior of the chemical reaction in the reactive system from data resulting from prior simulations or experiments, and by use of machine learning algorithms. Generally, these approaches that are not directly related to physics do not take into account all the physical phenomena involved in chemical reactions, in particular the conservation of mass of each atom present in the chemical species mixture. Such approaches therefore remain imprecise.

In order to take mass conservation into account, some approaches involve an additional step of checking mass conservation. However, this additional step is carried out outside the model, which limits optimization of the prediction.

Patent application WO/2020-176,914 describes a gas concentration prediction from neural networks. The method is applied to the post-treatment of engine exhaust gas. Direct calculation of the chemical kinetics is expensive, therefore resulting in the use of neural networks. However, for this method, the mass conservation is not accounted for the neural networks, which does not enable accurate prediction of the exhaust gas concentrations.

Patent application CN-109,918,702 describes the prediction of chemical states by use of neural networks, which accounts for the mass conservation of the atoms by use of a constraint. This constraint is however not included in the neural network.

“Hendricks et al., LINEARLY CONSTRAINED NEURAL NETWORKS, arXiv: 2002.01600, 2020” relates to the field of fluid mechanics. This document proposes an artificial neural network to guarantee a zero divergence of a velocity field in the case of an incompressible fluid and does not enable accounting for the mass conservation for a chemical reaction.

“Mohan et al., EMBEDDING HARD PHYSICAL CONSTRAINTS IN NEURAL NETWORK COARSE-GRAINING OF 3D TURBULENCE, arXiv: 2002.00021, 2020” relates to the field of fluid mechanics. It also proposes an artificial neural network to guarantee a zero divergence of a velocity field in the case of an incompressible fluid. It does not enable accounting for the mass conservation of a chemical reaction.

### SUMMARY OF THE INVENTION

The invention provides a simulation model of at least one chemical reaction allowing prediction, in a precise and reliable manner of the chemical compositions during the at least one chemical reaction. The present invention concerns a method of creating a simulation model of at least one chemical reaction, building an intermediate model by use of an artificial neural network and of a training base, and then creates the simulation model by adding an additional mass conservation layer to the artificial neural network.

Furthermore, the invention is a method of simulating a chemical reaction, implementing the simulation model.

The invention also relates to a method of creating a simulation model of at least one chemical reaction between several chemical species within a reactive system, using a training base, the training base comprising training input data and training output data of the chemical reaction, the training input and training output data are representative of the mass fractions of the chemical species at the inlet and at the outlet of the chemical reaction respectively. For this method, the following steps are carried out:
a. creating an intermediate model of the simulation model using an artificial neural network, comprising an input layer, at least one hidden layer and an output layer, creating the intermediate model being achieved using the training base, and
b. building the simulation model by use of the intermediate model and of an additional layer used after the output layer of the neural network, the additional layer having of a linear operator that applies a linear correction to the output data of the output layer of the artificial neural network to ensure mass conservation of the chemical elements forming the chemical species involved in the chemical reaction.

According to an embodiment, the additional layer is created by a step of linear correction calculation and of a step of applying the calculated linear correction.

Advantageously, the linear correction is calculated for chemical species of the chemical reaction corresponding to the number of chemical elements of the chemical reaction.

According to an implementation, synaptic weights of the artificial neural network are optimized after addition of the additional layer by use of the input and output data of the training base.

According to an aspect, the input and output data is determined before and after the chemical reaction respectively, or before and after a time interval of the chemical reaction respectively.

According to an embodiment option, the input data are mass fractions of the chemical species.

According to a feature, the output data are mass fractions of the chemical species or variations of the mass fractions of the chemical species.

Furthermore, the invention relates to a method of simulating at least one chemical reaction between chemical species within a reactive system. For this method, the following steps are carried out:
a. creating a simulation model by use of the method of creating a simulation model according to one of the above features; and
b. applying the simulation model to simulation input data, the simulation input data being representative of the mass fractions of the chemical species at the inlet of the chemical reaction.

According to an embodiment, the simulation model is created for a time interval, and the step of applying the simulation model is reiterated for successive time intervals, and, upon each reiteration, the simulation input data is the output data of the previous time interval.

According to an implementation, the chemical reaction is a chemical reaction within an energy conversion system which for example may be a combustion in an engine or a chemical reaction in an electric battery, or a chemical reaction in a fuel cell, or a chemical reaction in a system transporting the chemical species.

### BRIEF DESCRIPTION OF THE DRAWINGS

Other features and advantages of the method according to the invention are clear from reading the description hereafter of embodiments given by way of non-limitative example, with reference to the accompanying figures wherein:

FIG. 1 illustrates the steps of the method of creating a simulation model of a chemical reaction according to a first embodiment of the invention;

FIG. 2 illustrates the steps of the method of creating a simulation model of a chemical reaction according to a second embodiment of the invention;

FIG. 3 illustrates the steps of the method of simulating a chemical reaction according to a first embodiment of the invention;

FIG. 4 illustrates the steps of the method of simulating a chemical reaction according to a second embodiment of the invention;

FIG. 5 illustrates the steps of the method of simulating a chemical reaction according to a third embodiment of the invention;

FIG. 6 illustrates a chemical reaction simulation model according to an embodiment of the invention;

FIG. 7 illustrates a chemical reaction simulation model according to an embodiment of the invention;

FIG. 8 illustrates the mass fraction curves of four chemical elements as a function of time for a simulation implementing a method according to the prior art; and

FIG. 9 illustrates the mass fraction curves of four chemical elements as a function of time for a simulation implementing a method according to an embodiment of the invention.

### DETAILED DESCRIPTION OF THE INVENTION

The present invention relates to a method of creating a simulation model of at least one chemical reaction between several chemical species within a reactive system. The present invention relates to the prediction of chemical kinetics, that is the prediction of the chemical species during a chemical reaction, or at a given time of the chemical reaction.

By way of illustration only, the different terms are illustrated in the case of a combustion of methane in a combustion chamber. However, the method according to the invention can be applied to other chemical reactions. The various possible applications of the method according to the invention are described at the end of the present application.

A chemical reaction is understood to be the exchange of matter between various chemical species in a reactive system. Generally, the chemical reaction can be written as a balance equation. For the example of a combustion of methane (without taking account of the presence of nitrogen in the air), can be written as: CH₄+2O₂→CO₂+2H₂O.

The reactive system is the system wherein the chemical reaction occurs. For the example of the combustion of methane, the reactive system is the combustion chamber. The chemical species are understood to be the molecules involved in the chemical reaction. For the example for the combustion of methane, the chemical species are methane CH₄, oxygen O₂, carbon dioxide CO₂ and water H₂O. Furthermore, the chemical elements are understood to be the atoms involved in the chemical reaction. For the example for the combustion of methane, the chemical elements are carbon C, hydrogen H, oxygen O.

The present invention implements a training base. The training base comprises input data representative of the mass fractions of the chemical species at the inlet of the reactive system. The training base further comprises output data representative of the mass fractions of the chemical species at the reactive system outlet. The mass fraction of a chemical species is defined by the ratio of the mass of the chemical species to the total mass of the mixture in the reactive system.

The inlet and outlet can correspond to one of the following definitions: The inlet and outlet can correspond to a physical inlet and outlet of a system (before and after a chemical reaction), or to the evolution of the system between two time intervals during a numerical simulation (before or after a time interval of a chemical reaction).

Advantageously, the input data of the training base can correspond to the mass fractions of the chemical species.

According to an embodiment, the output data of the training base can correspond to the mass fractions of the chemical species.

As a variant, the output data of the training base can correspond to the variations of mass fraction of the chemical species. These variations are those achieved during the chemical reaction.

The input and output data of the training base can result from at least one of measured experimental data and from data obtained by simulation. According to an implementation of the invention, the method can comprise a preliminary step of creating the training base from at least one of the experimentally measured data and data obtained by simulation.

According to the invention, the method of creating a simulation model comprises the following steps:
1. Creating an intermediate model (artificial neural network)
2. Creating the simulation model.

These steps can be carried out by computing systems comprising notably a computer or a server. These steps are detailed in the rest of the description.

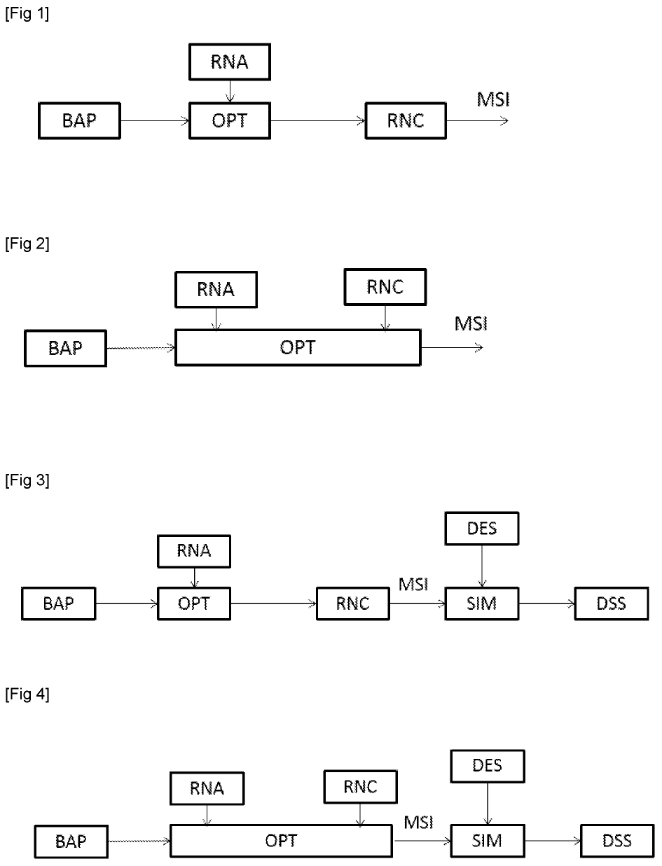

FIG. 1 schematically illustrates, by way of non-limitative example, the steps of the method according to a first embodiment of the invention. An intermediate model is formed from a training base BAP, by using an artificial neural network RNA and of an optimization OPT (training) of artificial neural network RNA. This optimization step allows determination of the synaptic weights of an artificial neural network RNA as a function of the data of training base BAP. The simulation model MSI is then built using the optimized (learned) artificial neural network RNA and an additional layer, referred to as additional conservation layer RNC.

According to an embodiment, the synaptic weights of the artificial neural network can be optimized after adding the additional conservation layer by using the data of the training base. In other words, training is applied to the entire model including the artificial neural network and the additional conservation layer. Therefore, the simulation model can be optimized by accounting for the data of the training base.

FIG. 2 schematically illustrates, by way of non-limitative example, the steps of the method of this embodiment. An artificial neural network RNA and additional conservation layer RNC are created. Then, from a training base BAP, artificial neural network RNA is optimized OPT (training) simultaneously with an additional conservation layer RNC to create the simulation model MSI. Thus, in this optimization step, an update of the synaptic weights of the artificial neural network with additional conservation layer RNC is performed from training database BAP.

Furthermore, the invention relates to a method of numerical simulation of at least one chemical reaction between chemical species within a reactive system. This numerical simulation method corresponds to a method of prediction of chemical species during a chemical reaction in a reactive system. The simulation method implements the model creating the method according to any one of the variants or variant combinations described in the present application, and a step of applying the simulation model to simulation input data. The numerical simulation then generates simulation output data.

The simulation input data and the simulation output data are similar to the input and output data of the training base. In other words, the simulation input and output data are representative respectively of the mass fractions of the chemical species at the inlet and at the outlet of the simulated reactive system.

The simulation inlet and outlet can correspond to one of a physical inlet and outlet of a system (before and after a chemical reaction), and the evolution of the system between two time intervals during a numerical simulation (before or after a time interval of a chemical reaction).

Advantageously, the simulation input data can correspond to the mass fractions of the chemical species.

According to an embodiment, the simulation output data can correspond to the mass fractions of the chemical species.

As a variant, the simulation output data can correspond to variations of fractions of the chemical species mass.

The simulation input data can be obtained experimentally or by simulation.

Thus, the simulation method comprises steps of:
1. Creating an intermediate model (artificial neural network)
2. Creating the simulation model
3. Applying the simulation model.

These steps can be carried out using a computer system, notably comprising a computer or a server. Preferably, steps 1 and 2 can be carried out beforehand only once, and step 3 can be repeated with the simulation model. These steps are detailed hereinafter.

FIG. 3 schematically illustrates, by way of non-limitative example, the steps of the numerical simulation method according to a first embodiment of the invention. From a training base BAP, an intermediate model is formed by using an artificial neural network RNA and an optimization OPT of artificial neural network RNA. This optimization step allows determination of the synaptic weights of artificial neural network RNA as a function of the data of training base BAP. Simulation model MSI is then built by using the optimized artificial neural network RNA and of an additional conservation layer RNC. Simulation model MSI is subsequently applied SIM to simulation input data DES in order to determine simulation output data DSS.

FIG. 4 schematically illustrates, by way of non-limitative example, the steps of the numerical simulation method according to a second embodiment of the invention. This second embodiment of the numerical simulation method implements the second embodiment of the simulation model building method of FIG. 2. An artificial neural network RNA and additional conservation layer RNC are created. Then, from a training base BPA, artificial neural network RNA is optimized OPT simultaneously with additional conservation layer RNC to create simulation model MSI. Thus, during this optimization step, an update of the synaptic weights of the artificial neural network with additional conservation layer RNC is performed from training database BAP. Simulation model MSI is subsequently applied SIM to simulation input data DES in order to determine simulation output data DSS.

According to an implementation of the invention, wherein a simulation is carried out for successive time intervals (that is for several intermediate intervals of the chemical reaction). The step applying the simulation model can be reiterated several times and, upon each reiteration, the simulation input data can be the simulation output data of the previous time interval. Thus, a chemical reaction can be simulated stepwise in an accurate and robust manner.

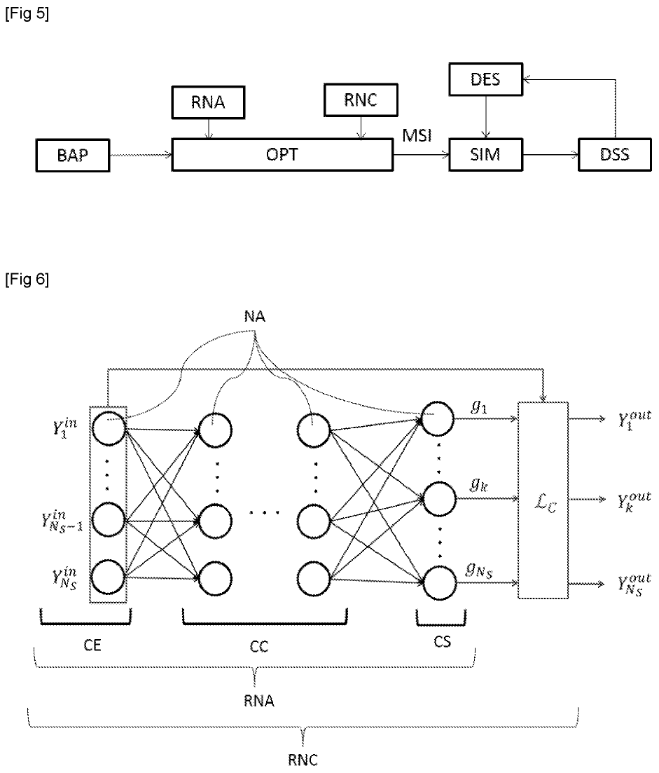

FIG. 5 schematically illustrates, by way of non-limitative example, the steps of the numerical simulation method according to a third embodiment of the invention. This third embodiment of the numerical simulation method implements the second embodiment of the simulation model method of FIG. 2. An artificial neural network RNA and additional conservation layer RNC are created. Then, from a training base BPA, artificial neural network RNA is optimized OPT simultaneously with additional conservation layer RNC to build simulation model MSI. Thus, during this optimization step, an update of the synaptic weights of the artificial neural network with additional conservation layer RNC is performed from training database BAP. Simulation model MSI is subsequently applied SIM to simulation input data DES in order to determine simulation output data DSS. The simulation step is then repeated for new simulation input data corresponding to the simulation output data of the previous time interval.

### 1. Creating the Intermediate Model

This step creates an intermediate model by using an artificial neural network. The model is referred to as being intermediate because it is not the simulation model obtained by the method. This intermediate model is modified according to step 2. The artificial neural network obtains a model enabling fast evaluation of the output data, with limited computer memory requirements, faster in particular than a complex numerical model representative of the chemical reaction. Moreover, the artificial neural network is suitable for complex chemical reactions, in particular those involving a large number of chemical species. Indeed, simulations of these complex systems can require significant computing systems, in particular in terms of memory, and significant simulation times. An artificial neural network is an algorithm whose parameters are optimized by use of a learning database from the training base, which contains the training input/output pairs. Once this network is trained on data, it can be used to perform inferences on input data never seen before.

The artificial neural network comprises an input layer, at least one hidden layer and an output layer. The input layer and the output layer comprise neurons corresponding to the number of chemical species of the chemical reaction. Creation of the artificial neural network is achieved using the training base. In other words, the synaptic weights of the neural network are learnt through supervised learning to correspond to the input and output data of the training base when formed, the intermediate model allows determination of output data from the input data, by being representative of the data of the training base. The inputs of the intermediate model are of the same type as the input data of the training base, and the outputs of the intermediate model are of the same nature as the output data of the training base.

The artificial neural network can take any form, for example a deep neural network, a recurrent neural network, a multilayer perceptron neural network, etc.

Building the intermediate model can involve a step of validation of the artificial neural network, notably to prevent overtraining problems. Validation can be performed using part of the training base data.

### 2. Creating the Simulation Model

This step creates the simulation model by using the intermediate model created in step 1 and of an additional layer referred to as additional conservation layer. The additional conservation layer is added after the output layer of the artificial neural network. The additional conservation layer is a linear operator for mass conservation of the chemical elements present in the chemical reaction. In other words, the simulation model comprises the input layer, the at least one hidden layer and the output layer of the artificial neural network, the additional conservation layer. Thus, the additional conservation layer allows determining the mass conservation of the chemical elements within the simulation model, which makes the simulation model more accurate. Furthermore, the linearity of the additional conservation layer allows maintaining the benefits of the artificial neural network which are simulation of complex systems with limited computer memory and limited computing time.

The additional conservation layer comprises a number of inputs and a number of outputs corresponding to the number of chemical species of the chemical reaction. Data representative of the mass fractions of the chemical species in the chemical reaction are determined at the output of the additional conservation layer and, therefore, at the output of the simulation model. Once determined, the additional conservation layer is static and constant. In other words, the additional conservation layer is not modified.

According to an aspect of the invention, the additional conservation layer can be created by use of a linear correction calculation step and a step of applying the calculated linear correction.

For this embodiment, in the case where the number of chemical species is greater than the number of chemical elements, the correction step can be applied for a number of chemical species corresponding to the number of chemical elements. Preferably, the chemical species selected can contain at least once each atom present in the mixture. Thus, underdetermination of the problem can be avoided.

According to an embodiment wherein the input data and the output data is normalized, these two steps (correction and correction application) can be preceded by a normalization inversion step, followed by a normalization step.

These two steps (correction and correction application) are detailed in the rest of the description below.

Hereinafter a mixture of chemical species numbered 1, . . . , Ns, and described by the mass fraction Yₖ of each of these species are considered. The mass fraction is defined by Yₖ=mₖ/m, where mₖ is the mass of the species k in the mixture and m is the total mass of the mixture. The chemical species are considered to react with each other, by excluding nuclear type reactions. Therefore, the mass of each atom making up the molecules of the mixture is conserved between the input state and the output state. In the rest of the description, a chemical input state is denoted by in and the corresponding output state by out. The corresponding mass fractions are denoted by Yₖⁱⁿ and Yₖ^{out} respectively. The change between input and output involves a certain number of chemical reactions between these species.

Any atom (chemical element) present in the mixture is denoted by j (for example: j=0, H, C, N in the case of a system involving combustion reactions).

It is assumed that there are Nₐ distinct atoms in the mixture. Fraction Yⱼ corresponds to the mass of atom j in relation to the total mass of the mixture. This mass fraction can be written from the mass fractions of species as follows:

$${Y}_{j} = {\sum }_{k = 1}^{{N}_{S}} \frac{{M}_{j}}{{M}_{k}} {n}_{k}^{j} {Y}_{k}$$

where Mₖ corresponds to the molar mass of chemical species k, nₖ^{j} is the number of atoms j present in the molecule of chemical species k, and Mⱼ corresponds to the molar mass of atom j. This relation can be written in matrix form by putting Yₐ∈ [symbol] which is the vector containing the mass fractions of the atoms and Yₛ∈ [symbol] which is the vector of the mass fractions of the chemical species. It is expressed as:

Yₐ= [symbol] Yₛ

with: [symbol] =(mⱼₖ) such that

$$∀ j , k , {m}_{jk} = \frac{{M}_{j}}{{M}_{k}} {n}_{k}^{j} .$$

The conservation of mass of the atoms between the input and the output gives the following property, which can be expressed for any atom j present in the mixture:

Yⱼⁱⁿ=Yⱼ^{out}

i.e., in matrix form:

[symbol]

Yₛⁱⁿ= [symbol] Yₛ^{out}

The output vector of the artificial neural network is denoted by g. This embodiment corrects vector g to satisfy the previous relation. The correction can be written by adding an a priori term to be determined, denoted by α:

Yₛ^{out}=g+α

With α∈ [symbol] being a vector allowing vector g to be corrected. The previous equation becomes:

[symbol]

α= [symbol] (Yₛⁱⁿ−g)

This equation is a linear system with unknowns αₖ, k∈ [symbol] 1, Nₛ [symbol] . Insofar as [symbol] ∈ [symbol] , with the system containing Nₛ unknowns and Nₐ equations. In general, the number of atoms is well below the number of species (Nₐ<<Nₛ), and the system is therefore underdetermined.

In order to remove the underdetermination, a set of Nₐ chemical species from among the Nₛ present in the mixture can be extracted. It is preferable that these species contain at least once each atom present in the mixture. The subscripts of these species are denoted by l₁, . . . , l_{N}_{ₐ}. It is assumed that αₖ≠0∀k∈{l₁, . . . , l_{N}_{ₐ}} and αₖ=0 otherwise (in other words, for this implementation, a correction can be provided for the chemical species selected, and no correction is made for the other species of the chemical reaction). A new correction vector can be defined:

$${α}^{′} = ( \begin{matrix}{α}_{{l}_{1}} \\ ⋮ \\ {α}_{{l}_{{N}_{a}}}\end{matrix} )$$

The system of equations then becomes:

[symbol]

α′= [symbol] (Yₛⁱⁿ−g)

where [symbol] corresponds to the submatrix of [symbol] containing only the columns corresponding to species l₁, . . . , l_{N}_{ₐ}. This results in [symbol] ∈ [symbol] Moreover, if each atom is represented in the species selected, it is possible to show that the system is invertible. The solution is then denoted by:

α′= [symbol] ( [symbol] (Yₛⁱⁿ−g)

Thus, with this calculation, the correction is determined. Then, by applying this correction to the species selected, the exact conservation of the atoms is guaranteed. This correction application can be written for each chemical species k:

yₖ^{out}=g+α

According to an implementation of the invention (corresponding to FIG. 2), a modification of the neural network, in the present case a modification of the synaptic weights, can be provided after applying the correction. Thus, this correction is applied within the neural network, the parameters of the at least one hidden layer upstream can be adjusted so that the output is well predicted, despite the fact that an arbitrarily chosen set of species is selected to correct the mass of the atoms.

FIG. 6 schematically illustrates, by way of non-limitative example, the building of the simulation model according to an embodiment of the invention. Artificial neural network RNA is first created. Artificial neural network RNA comprises an input layer CE for which the mass fractions of chemical species Y₁ⁱⁿ, . . . , Y_{Ns}ⁱⁿ form the input data. Artificial neural network RNA comprises at least one hidden layer CC. Artificial neural network RNA further comprises an output layer CS for which the mass fractions of chemical species g₁, . . . , g_{Ns} form the output data. The layers of artificial neural network RNA comprise artificial neurons NA represented by circles. A synaptic weight is associated with each artificial neuron NA. In addition, simulation model RNC further comprises an additional conservation layer [symbol] _{c} that determines the linear correction of output data g₁, . . . , g_{Ns} of artificial neural network RNA to determine the mass fractions of chemical species Y₁^{out}, . . . , Y_{Ns}^{out} at the output, while ensuring the mass conservation of the chemical elements. For the embodiment of FIG. 6, for which output data Y₁^{out}, . . . , Y_{Ns}^{out} are mass fractions and additional conservation layer [symbol] _{c} accounts for input data Y₁ⁱⁿ, . . . , Yᵢⁱⁿ.

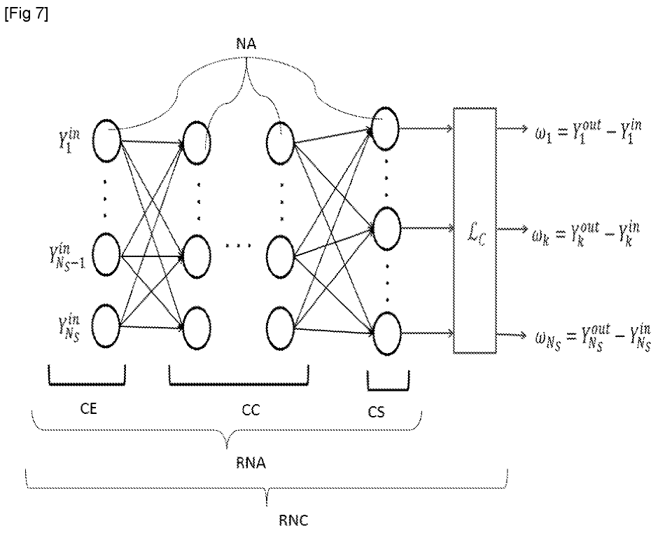

FIG. 7 is similar to FIG. 6, in the case where the output data are variations of the mass fraction of chemical species ω₁, . . . , ω_{Ns}, with ωᵢ=Y₁^{out}−Yᵢⁱⁿ Elements identical to FIG. 6 are not described again. For this embodiment, additional conservation layer [symbol] _{c} does not need to account for input data Y₁ⁱⁿ, . . . , Y_{Ns}ⁱⁿ.

### 3. Applying the Simulation Model

This step only concerns the numerical simulation method for at least one chemical reaction. In this step, the simulation model created in step 2 is applied to simulation input data. In other words, the chemical kinetics of the chemical reaction is simulated from simulation input data and from the simulation model. It may be a simulation for the entire chemical reaction or for a time interval of the chemical reaction.

According to an implementation of the invention, for which a simulation is performed for successive time intervals (that is for several intermediate steps of the chemical reaction), the step of applying the simulation model can be reiterated several times and, upon each reiteration, the simulation input data can be the simulation output data of the previous time interval. Thus, a chemical reaction can be simulated stepwise, in an accurate and robust manner. This implementation corresponds to that illustrated in FIG. 5.

The chemical reaction can be a chemical reaction in the field of reactive fluid mechanics. In this case, the chemical species can be liquids or gases.

According to a first example implementation, the invention can concern a chemical reaction of a combustion, in particular in a combustion engine, a turbine, or any similar system. Indeed, the methods according to the invention can enable modeling of complex systems with many chemical species.

According to a second example implementation, the invention can concern a chemical reaction in an energy storage and supply system, in particular in an electric battery or a fuel cell. Indeed, the methods according to the invention can enable modeling of complex systems with many chemical species.

According to a third example implementation, the invention can concern a catalytic chemical reaction, in particular a catalysis within an exhaust gas post-treatment system, or a catalysis in the field of hydrocarbon production. Indeed, the methods according to the invention can enable modeling of complex systems with many chemical species.

According to a fourth example, the invention can concern a chemical reaction specific to fluid flows in porous media, in particular a reaction of precipitation or dissolution of one or more minerals. Indeed, the methods according to the invention can enable modeling of complex systems with many chemical species.

Furthermore, the invention relates to a numerical chemical reaction simulator implementing the simulation model created by the method according to one of the variants or variant combinations of the method according to the invention. The numerical simulator uses simulation input data to determine simulation output data. The simulation input data and the simulation output data is representative of the mass fractions of the chemical species of the chemical reaction.

In addition, the invention relates to a method of designing a reactive system in which at least one chemical reaction takes place, wherein the following steps are carried out:
numerically simulating the chemical reaction in the reactive system by using the simulation method according to any one of the variants or variant combinations described above, and
designing the reactive system and carrying out the chemical reaction in the reactive system.

For this design method, numerical simulations can be performed for chemical reactions (by at least one of modifying the chemical species initially present for example) and the reactive systems (for several reactive system dimensions or shapes for example). Comparison of the simulations allows determination of at least one of chemical reaction and the reactive system to be designed.

The present invention can also relate to a method of controlling a reactive system wherein at least one chemical reaction takes place and wherein the following steps are carried out:
numerically simulating the chemical reaction in the reactive system by using the simulation method according to any one of the variants or variant combinations described above; and
controlling the reactive system, preferably in real time, as a function of the simulated chemical reaction, notably in order to avoid dangerous situations, or situations not suitable for the desired use.

Preferably, to control the reactive system, data used as simulation input data can be measured in real time.

For the example of a combustion, control the amount of air or of fuel in the combustion chamber. The purpose of this control can be to limit pollutant emissions or to prevent knock phenomena, etc.

For the example of an electrical battery, control can correspond to controlling the voltage or the current, notably in order to limit overheating or premature aging of the electric battery.

For the example of a fuel cell, control can notably correspond to controlling the air flow supply to optimize the chemical kinetics within the fuel cell and thereby to improve the performances thereof.

### Comparative Example

The features and advantages of the method according to the invention will be clear from reading the comparative example hereafter.

To illustrate the invention, an example is given in this part. Here the use of neural networks in numerical simulation of a combustion is considered. Here is an exact resolution algorithm as replaced with an equivalent neural network. In particular, the neural network takes as inputs mass fractions at a time t of the simulation (state in in the description of the method above) and it assesses the mass fractions at a time t+dt (state out). This is performed iteratively for tₖ=kdt, k∈[0, Nᵢₜₑ], where Nᵢₜₑ is the total number of iterations of the simulation.

The case considered here is a case of methane combustion at constant pressure, where the atoms generally present are carbon (C), hydrogen (H), oxygen (O) and nitrogen (N).

For this comparative example, a method according to the prior art implementing an artificial neural network (without an additional conservation layer) and a method according to the invention implementing an artificial neural network with an additional conservation layer are compared. For the two simulations, the training base is the same, and the number of layers and the number of neurons of the artificial neural network are the same. More particularly, the selected neural network is a multilayer perceptron type network comprising two hidden layers with 64 units each. The database is simulations of the combustion of H₂/air mixtures for different initial temperatures T₀ and fuel/air ratios ϕ. A thousand simulations are selected in the training base by use of a Latin hypercube sampling algorithm. This sampling is performed for values of T₀ between 1600 K (approximately 1326° C.) and 1800 K (approximately 1526° C.), and fuel/air ratio values between 0.7 and 1.5. Optimizing the weights of the network is done using a gradient descent method.

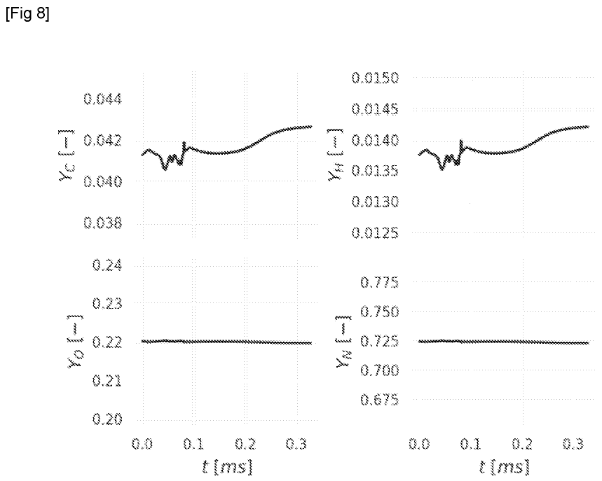

FIG. 8 illustrates the curves of the method according to the prior art, as a function of time t in ms, of the normalized carbon mass fraction Y_{C} (top left), the normalized hydrogen mass fraction Y_{H} (top right), the normalized oxygen mass fraction Y_{O} (bottom left) and the normalized nitrogen mass fraction Y_{N} (bottom right). It is noted in these figures that the carbon and hydrogen mass fraction curves are not constant over time. Therefore, the artificial neural network according to the prior art does not enable the mass conservation of the carbon atoms and the hydrogen atoms, while the mass conservation of atoms during simulation is a critical point. Indeed, a deviation of the mass at a given time can have a negative impact on the rest of the simulation and make the simulation inaccurate.

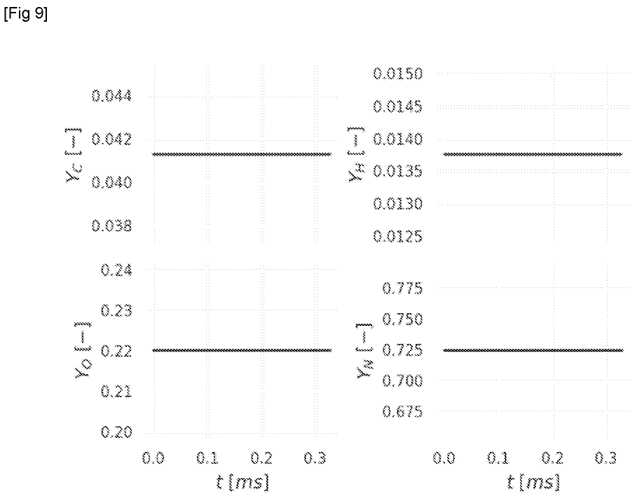

FIG. 9 illustrates the curves of the method according to the invention (with the additional conservation layer), as a function of time t in ms, of the normalized carbon mass fraction Y_{C} (top left), the normalized hydrogen mass fraction Y_{H} (top right), the normalized oxygen mass fraction Y_{O} (bottom left) and the normalized nitrogen mass fraction Y_{N} (bottom right). It is noted that the four curves are constant over time. Therefore, using layer [symbol] _{c} according to the invention provides mass conservation of the atoms, hence higher accuracy and robustness of the simulation model.

1-10. (canceled)

11. A method of creating a simulation model of at least one chemical reaction between chemical species within a reactive system, using a training base, the training base comprising training input data and training output data of the chemical reaction, the training input and training output data being representative of mass fractions of the chemical species at an inlet and at an outlet of the chemical reaction respectively, comprising:
a.) creating an intermediate model of the simulation model using an artificial neural network comprising an input layer, at least one hidden layer and an output layer and creating the intermediate model using the training base; and
b.) creating the simulation model by using the intermediate model and using an additional layer after the output layer of the neural network, the additional layer being a linear operator applying a linear correction to the output data of the output layer of the artificial neural network to ensure mass conservation of chemical elements within the reactive system of the chemical species involved in the at least one chemical reaction.

12. A method of creating a simulation model as claimed in claim 11, wherein the additional layer is created by using a linear correction calculation which is applied to the output data of the output layer.

13. A method of creating a simulation model as claimed in claim 12, wherein the linear correction is calculated for a number of chemical species of the chemical reaction corresponding to the number of chemical elements used in the chemical reaction.

14. A method of creating a simulation model as claimed in claim 11, wherein synaptic weights of the artificial neural network are optimized after adding the additional layer by using the input and output data of the training base.

15. A method of creating a simulation model as claimed in claim 12, wherein synaptic weights of the artificial neural network are optimized after adding the additional layer by using the input and output data of the training base.

16. A method of creating a simulation model as claimed in claim 13, wherein synaptic weights of the artificial neural network are optimized after adding the additional layer by using the input and output data of the training base.

17. A method of creating a simulation model as claimed in claim 11, wherein the input and output data is determined before and after the chemical reaction or before and after a time interval of the chemical reaction.

18. A method of creating a simulation model as claimed in claim 11, wherein the input data are mass fractions of the chemical species involved in the at least one chemical reaction.

19. A method of creating a simulation model as claimed in claim 11, wherein the output data are mass fractions of the chemical species or variations of the mass fractions of the chemical species involved in the at least one chemical reaction.

20. A method of simulating at least one chemical reaction between chemical species within a reactive system, comprising:
a.) creating a simulation model by using the method of creating a simulation model in accordance with claim 11; and
b.) creating the simulation model to simulation of input data, the simulation input data being representative of mass fractions of the chemical species at the inlet of the chemical reaction.

21. A method of simulation as claimed in claim 20, wherein the simulation model is created for a time interval, and applying the simulation model is reiterated for successive time intervals, and, upon each reiteration, the simulation input data is the simulation output data of a previous time interval.

22. A simulation method as claimed in claim 20, wherein the chemical reaction is a chemical reaction within an energy conversion system comprising one of combustion in an engine, a chemical reaction in an electric battery, a chemical reaction in a fuel cell, and a chemical reaction in a system of transporting the chemical species.
